(19) United States
(12) Patent Application Publication
(10) Pub. No.: US 2021/0214769 A1
(43) Pub. Date: Jul. 15, 2021

(54) METHODS AND SYSTEMS FOR AMPLIFYING LOW CONCENTRATIONS OF NUCLEIC ACIDS

(71) Applicant: Fluent Biosciences Inc., Watertown, MA (US)

(72) Inventors: Kristina Fontanez, Arlington, MA (US); Robert Meltzer, Belmont, MA (US); Yi Xue, Shrewsbury, MA (US); Sepehr Kiani, Watertown, MA (US)

(21) Appl. No.: 17/146,757

(22) Filed: Jan. 12, 2021

Related U.S. Application Data
(60) Provisional application No. 62/960,399, filed on Jan. 13, 2020.

(51) Int. Cl. C12Q 1/6806; C12Q 1/6844; C12Q 1/6886

(52) U.S. Cl. CPC C12Q 1/6806; C12Q 2600/156; C12Q 1/6886; C12Q 1/6844

(57) ABSTRACT

This disclosure provides methods and systems for amplifying small amounts of nucleic acids. Methods include performing amplification reactions in an emulsion format to isolate and clonally amplify discrete populations of nucleic acid molecules inside droplets. In particular, methods and systems of the invention generate an emulsion with particles that template the formation of droplets inside a tube and segregate nucleic acid molecules therein such that each droplet contains an individual nucleic acid molecule. The nucleic acid molecules are amplified inside the droplets.

### TECHNICAL FIELD

This disclosure relates to methods and systems for amplifying low concentrations of nucleic acids.

### BACKGROUND

The ability to amplify low concentrations of nucleic acids is important for the early detection and treatment of cancer. For example, clinicians can amplify trace amounts of cell free DNA (cfDNA) collected from blood biopsies to look for genetic mutations that are indicative of cancer. This provides a non-invasive approach to identify types and stages of cancer and monitor its progression throughout a treatment regimen.

Unfortunately, the amount of cfDNA that can be collected from a blood biopsy is low, and at low concentrations, methods of nucleic acid amplification are sensitive to losses during sample preparation and are prone to amplification biases that result in reduced data quality. These drawbacks impact the ability of clinicians to accurately interrupt data collected from cfDNA analysis, which results in undetected cancers and ineffective treatments.

### SUMMARY

This disclosure provides methods and systems for amplifying small amounts of target nucleic acids. Methods include performing amplification reactions in an emulsion format to isolate and clonally amplify discrete populations of nucleic acid molecules inside droplets. In particular, methods and systems of the invention generate an emulsion with particles that template the formation of droplets inside a tube and segregate nucleic acid molecules therein such that each droplet contains an individual nucleic acid molecule. The nucleic acid molecules are amplified inside the droplets. Thus, each droplet functions as an isolated reaction chamber, compartmentalizing the reaction into multiple parallel reactions. The emulsion format ensures that every nucleic acid molecule has equal access to resources required for amplification. Thus, amplification biases are reduced by eliminating interferences among nucleic acid molecules. Moreover, because methods of the invention can be used to collect and amplify nucleic acid molecules in a single reaction tube, material loss during sample preparation is reduced.

Methods and systems of the invention provide a method for amplifying nucleic acid molecules present at low concentrations. The method includes combining template particles and nucleic acid molecules in an aqueous fluid within a vessel, wherein the concentration of the nucleic acid molecules inside the vessel is at least below 1 ng per μl (e.g., at least below 0.5 ng, at least below 0.1 ng, at least below 0.01 ng, or at least below 1 pg). The method further includes adding a second fluid to the vessel and vortexing the vessel to form a plurality of monodisperse droplets simultaneously wherein each of the nucleic acid molecules are isolated within a separate one of the monodisperse droplets. Preferably, vortexing the vessel involves putting the vessel onto a vortexer. Methods of the invention further include amplifying the nucleic acid molecules contained inside the droplets to generate a plurality of amplicons that can be analyzed by various methods for certain clinical, research, and forensic applications.

Methods provided by this disclosure can amplify a target even where the target is present only in very small quantities, e.g., as low as 0.01% frequency of total fragments in a given sample. Such methods will have particular applicability for the amplification of cfDNA, which is present in present in blood at approximately 10-1000 fragments per mL. Thus, methods provided by the invention may provide genetic material that can be used to discover very rare yet clinically important information, such as, mutations that are specific to a tumor.

In certain aspects, methods and systems of the invention provide a method for amplifying cfDNA. The method includes obtaining a sample containing a cfDNA fragment in blood or plasma and combining template particles and at least of portion of the sample in an aqueous fluid within a vessel. In some instances, the vessel may comprise a blood collection tube containing certain reagents for sample preservation or nucleic acid synthesis. Methods further include adding a second fluid to the vessel and vortexing the vessel to form a plurality of monodisperse droplets simultaneously such that the cfDNA fragment is isolated within one of the droplets. The cfDNA is then amplified inside the droplet.

In some instances, the cfDNA is circulating tumor DNA (ctDNA), derived from a tumor or circulating tumor cell. The ctDNA may include one or more mutations associated with cancer. Methods of the invention can be used to isolate and amplify fragments of ctDNA collected from a blood sample to generate a plurality of amplicons derived from the ctDNA fragment. Methods of the invention may further include analyzing the amplicons to provide genetic information from the subject, wherein the genetic information describes one or more mutations in a subject.

In some embodiments, the target nucleic acid includes a mutation specific to a tumor. The target nucleic acid may be present at no more than about 0.01% of cell-free DNA in the plasma or serum. By methods herein, the target nucleic acid is isolated from a plurality of nucleic acids for amplification. In some instances, the methods may further include detecting the target nucleic acid (e.g., by sequencing, probe hybridization, qPCR, digital PCR, etc.). For example, detecting the target nucleic acid may include hybridizing the target nucleic acid to a probe or to a primer for a detection or amplification step, or labelling the target nucleic acid with a detectable label.

In other aspects, methods of the invention include quantifying target nucleic acid molecules amplified inside monodisperse droplets. Methods for quantification include PCR-based strategies, such as, qPCR, RT-qPCR, or single molecule counting using digital PCR. The number and nature of primers used in such assays may vary, based at least in part on the type of assay being performed. For example, in some instances, methods may include using primers to detect specific genes (e.g., oncogenes). Alternatively, the amplicons may be quantified by sequencing.

In certain aspects, methods and systems of the invention provide a method for isolating a target nucleic acid from a plurality of nucleic acids by segregating the nucleic acids into separate droplets. The droplets may be prepared as emulsions, e.g., as an aqueous phase fluid dispersed in an immiscible phase carrier fluid (e.g., a fluorocarbon oil, silicone oil, or a hydrocarbon oil) or vice versa. Generally, the droplets are formed by shearing two liquid phases. Shearing may comprise any one of vortexing, shaking, flicking, stirring, pipetting, or any other similar method for mixing solutions.

Methods and systems of the invention use template particles to template the formation of monodisperse droplets and segregate nucleic acids therein. Template particles according to aspects of the invention may comprise hydrogel, for example, selected from agarose, alginate, a polyethylene glycol (PEG), a polyacrylamide (PAA), acrylate, acrylamide/bisacrylamide copolymer matrix, azide-modified PEG, poly-lysine, polyethyleneimine, and combinations thereof. In certain instances, template particles may be shaped to provide an enhanced affinity a nucleic acid. For example, the template particles may be generally spherical but the shape may contain features such as flat surfaces, craters, grooves, protrusions, and other irregularities in the spherical shape that promote an association with a nucleic such that the shape of the template particle increases the probability of templating a monodisperse droplet that contains a nucleic acid.

In some aspects, methods and systems of the invention provide template particles that include one or more internal compartments. The internal compartments may contain a reagent or compound that is releasable upon an external stimulus. Reagents contained by the template particle may include, for example, nucleic acid synthesis reagents (e.g., a DNA polymerase). The external stimulus may be heat, osmotic pressure, or an enzyme. For example, in some instances, methods of the invention include introducing a polymerase inside a monodisperse droplet and performing a PCR reaction therein.

### BRIEF DESCRIPTION OF DRAWINGS

FIG. 1 diagrams a method for amplifying target nucleic acids.

FIG. 2 shows a vessel containing nucleic acids and template particles before vortexing.

FIG. 3 shows a vessel containing nucleic acids and template particles inside droplets.

### DETAILED DESCRIPTION

This disclosure provides methods and systems for amplifying nucleic acids present at a low concentration in a sample. Methods use an emulsion format to isolate and clonally amplify discrete populations of nucleic acid molecules inside droplets. In particular, methods and systems of the invention generate an emulsion with particles that template the formation of droplets inside a tube and segregate nucleic acid molecules therein such that each droplet contains an individual nucleic acid molecule. The nucleic acid molecules are amplified inside the droplets. Thus, each droplet functions as an isolated reaction chamber, compartmentalizing the reaction into multiple parallel reactions. The emulsion format ensures that every nucleic acid molecule has equal access to resources required for amplification. Thus, amplification biases are reduced by eliminating interferences among nucleic acid molecules. Moreover, because methods of the invention can be used to collect and amplify nucleic acid molecules in a single reaction tube, material loss during sample preparation is reduced.

The droplets all form substantially simultaneously at a moment of shearing immiscible fluids. Each droplet provides an aqueous partition, surrounded by oil. An important insight of the disclosure is that template particles can modulate an environment for amplification by forming reaction chambers with defined volumes of aqueous solution and suitable concentrations of nucleic acid synthesis reagents. Moreover, the reagents, such as, DNA polymerase, may be delivered directly into droplets via the template particles to ensure each droplet receives a substantially uniform quantity of reagents.

Methods of the invention can be used to amplify a target even where the target is present only in very small quantities, e.g., even as low as 0.01% frequency total fragments in a given sample. Thus, methods of the invention may have particular applicability for the amplification of cfDNA, which is DNA that is freely circulating in the bloodstream and only present in blood at very low concentrations. Faithful amplification of cfDNA, as provided by methods of the invention, provides genetic material that can be used for a variety purposes as cfDNA has been shown to be a useful biomarker for a multitude of ailments including cancer and fetal medicine. This includes but is not limited to trauma, sepsis, aseptic inflammation, myocardial infarction, stroke, transplantation, diabetes, and sickle cell disease.

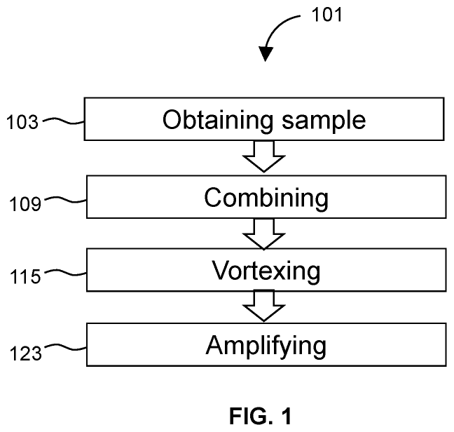

FIG. 1 diagrams a method 101 for amplifying target nucleic acids. The target nucleic acid may be either DNA or RNA, whose nucleotides are linked together to form a chain. Methods of the invention are particularly well suited for amplifying nucleic acids of low concentrations. In some instances, the nucleic acid target comprises free circulating DNA (cfDNA), which encompasses cell-free fetal DNA, cell-free tumor DNA, and cell-free circulating mitochondrial DNA.

In some embodiments, the method 101 includes obtaining 103 a sample comprising cfDNA from blood or plasma. Obtaining 103 the sample may include performing a blood draw to obtain blood or receiving blood from a clinical facility. A 10 ml sample of blood may contain only about 1 ng of cfDNA. Thus as used herein, a sample may be a blood sample drawn (e.g. with a needle) from a peripheral blood source (e.g. an arm vein) from a patient. Before use in methods of the invention, the sample of peripheral blood can be treated with an agent to inhibit blood coagulation, such as heparin. Preferably, the blood sample contains at least approximately 1 ng of cfDNA.

In some embodiments, obtaining 103 a sample involves a phlebotomy procedure and collects blood into blood collection tube such as the blood collection tube sold under the trademark VACUTAINER by BD (Franklin Lakes, N.J.) or a cell-free DNA blood collection tube such as that sold under the trademark CELL-FREE DNA BCT by Streck, Inc. (La Vista, Nebr.). Any suitable collection technique or volume may be employed.

After obtaining 103 the sample, subsequent steps of the method 101 may be performed. Alternatively, the concentration of target cfDNA in the sample may be enriched. Enrichment of the targets inside the sample may be done by any method known in the art, for example, by centrifugation on a density gradient.

After obtaining 103 the sample, the sample is combined 109 with template particles, an aqueous fluid inside a vessel and an oil. Combining 109 may include a variety of suitable methods in various orders. One method for combining 109 is to use a pipette to draw up a portion of the sample containing cfDNA suspended in an aqueous fluid and dispense the sample into a vessel containing template particles, for example, a small sample preparation tube, such as, the sample preparation tube sold under the name Eppendorf, or, a 15 ml conical tube, such as the conical tube sold under the tradename Falcon. In some instances, the vessel may be a blood collection tube, such as the blood collection tube sold under the name Vacutainer, in which the template particles are disposed—preferably in a dried format. Combining 109 is then be accomplished by obtaining 103 the sample via blood draw directly into the blood collection tube and then adding an oil.

The method 101 then includes vortexing the vessel. Vortexing 109 is preferably done by pressing the vessel onto a vortexer, which creates sufficient shear forces inside the vessel to partition the aqueous fluid into monodisperse droplets. After vortexing 109, a plurality monodisperse droplets (e.g., at least 100, at least 1,000, at least 1,000,000, at least 10,000,000 ore more) is formed essentially simultaneously. At least one of the droplets will have at least one cfDNA fragment and a template particle.

After vortexting, 109 the cfDNA fragments are amplified 123 inside of the droplets. Various methods or techniques can be used to amplify 123 the isolated cfDNA fragments, for example, as discussed in WO 2019/139650, and WO 2017/031125, which are both incorporated by reference. Preferably, amplifying 123 is accomplished by PCR to generate amplicons of the cfDNA fragments. The amplicons may be stored or analyzed by, for example, sequencing.

In some instances, amplifying 123 may occur by nonspecific amplification methods. For example, primers containing random sequences may be used. In other instances, sequence-specific amplification methods are used. Therefore, in some embodiments, amplification 123 reactions include one or more primers. For example, in some embodiments, each droplet may comprises at least 20 primer pairs. In some embodiments, each droplet may comprise at least 50 primer pairs. In some embodiment, each droplet may comprise at least 200 primer pairs. In some embodiments, each droplet may comprise at least 500 primer pairs.

In some embodiments, a target gene or gene region for amplification is a gene or gene region having a rare mutation. In some embodiments, a target gene or gene region for amplification is a gene or gene region that is associated with a cancer or an inherited disease. For example, primers may be designed as to amplify specific genes of interest which include, but are not limited to, BAX, BCL2L1, CASP8, CDK4, ELK1, ETS1, HGF, JAK2, JUNB, JUND, KIT, KITLG, MCL1, MET, MOS, MYB, NFKBIA, EGFR, Myc, EpCAM, NRAS, PIK3CA, PML, PRKCA, RAF1, RARA, REL, ROS1, RUNX1, SRC, STAT3, CD45, cytokeratins, CEA, CD133, HER2, CD44, CD49f, CD146, MUC1/2, ABL1, AKT1, APC, ATM, BRAF, CDH1, CDKN2A, CTNNB1, EGFR, ERBB2, ERBB4, EZH2, FBXW7, FGFR2, FGFR3, FLT3, GNAS, GNAQ, GNAl1, HNF1A, HRAS, IDH1, IDH2, JAK2, JAK3, KDR, KIT, KRAS, MET, MLH1, NOTCH1, NPM1, NRAS, PDGFRA, PIK3CA, PTEN, PTPN11, RB1, RET, SMAD4, STK11, TP53, VHL, and ZHX2.

In some embodiments, a primer used in an amplification reaction can be attached to a surface of a template particle. In some embodiments, a surface of the template particle can comprise a plurality of primers. In other embodiments, some primers are not attached to the template particles and rather are included in an aqueous fluid and are segregated into the monodisperse droplets upon shearing the mixture. In other embodiments, primers are delivered into the droplets via compartments within the particle templates.

In some aspects, non-PCR based DNA amplification techniques may be used. For example, in some instances multiple displacement amplification (MDA) methods can be used to amplify target nucleic acids inside droplets. For example, see U.S. Pat. No. 6,124,120, which is incorporated by reference. MDA amplification may have advantages over the PCR-based methods since MDA amplification can be carried out under isothermal conditions. No thermal cycling is needed because the polymerase at the head of an elongating strand (or a compatible strand-displacement protein) will displace, and thereby make available for hybridization, the strand ahead of it. Other advantages of multiple strand displacement amplification include the ability to amplify very long nucleic acid segments (on the order of 50 kilobases) and rapid amplification of shorter segments (10 kilobases or less). In multiple strand displacement amplification, single priming events at unintended sites will not lead to artefactual amplification at these sites (since amplification at the intended site will quickly outstrip the single strand replication at the unintended site)

Methods of the invention include preparing a target fragment of nucleic acid inside a droplet for sequencing. Methods may include barcoding target fragments to prepare for downstream sequencing analysis. Any suitable methods may be used to barcode target fragments inside droplets for sequencing. Suitable approaches to attached barcodes to target fragments may include (i) fragmentation and adaptor-ligation (in which adaptors include barcodes); (ii) tagmentation (using transposase enzymes or transpososomes including those sold in kits such as those tagmentation reagent kits sold under the trademark NEXTERA by Illumina, Inc.); and (iii) amplification by, e.g., polymerase chain reaction (PCR) using primers with a hybridization portion complementary to a known or suspected target of interest in a genome and at least one barcode portion that is copied into the amplicons by the PCR reaction. For any of these approaches, the barcodes (e.g., within amplification primers or ligatable adaptors) may be provided free an in solution or bound to a template particle as described herein. In some embodiments, the barcodes are provided as a set (e.g., including thousands of copies of a barcode) in which each barcode is covalently bound to a template particle.

As used herein, barcode generally refers to an oligonucleotide that includes an identifier sequence that can be used to identify sequence reads originating from target nucleic acids that were barcoded as a set with copies of one barcode unique to that set. Barcodes generally include a known number of nucleotides in the identifier sequence between about 2 and about several dozen or more. The oligonucleotides that include the barcodes may include any other of a number of useful sequences including primer segments (e.g., designed to hybridize to a target of interest in a genetic material), universal primer binding sites, restriction sites, sequencing adaptors, sequencing instrument index sequences, others, or combinations thereof. For example, in some embodiments, barcodes of the disclosure are provided within sequencing adaptors such as within a set of adaptors designed for use with a next generation sequencing (NGS) instrument such as the NGS instrument sold under the trademark HISEQ by Illumina, Inc. Within an NGS adaptor, the barcode may be adjacent the index portion or the target sequence such that the barcode sequence is found in the index read or the sequence read.

In some aspects, a template particle may include capture oligos with portions that hybridize or ligate to the fragment of nucleic acid. The capture oligos may include gene-specific sequences or random sequences that hybridize to the target fragment by complementary base pairing. In other instances, the capture oligo may be ligated onto a first end or a second end of the target fragment. The capture oligos may include a binding site sequence P5, and an index. The capture oligos may further include a binding sequence P7 and a hexamer. Any suitable sequence may be used for the P5 and P7 binding sequences. For example, either or both of those may be arbitrary universal priming sequence (universal meaning that the sequence information is not specific to the naturally occurring genomic sequence being studied, but is instead suited to being amplified using a pair of cognate universal primers, by design). The index segment may be any suitable barcode or index such as may be useful in downstream information processing. It is contemplated that the P5 sequences, the P7 sequence, and the index segment may be the sequences use in NGS indexed sequences such as performed on an NGS instrument sold under the trademark ILLUMINA, and as described in Bowman, 2013, Multiplexed Illumina sequencing libraries from picogram quantities of DNA, BMC Genomics 14:466, incorporated by reference. The hexamer segments may be random hexamers or selective hexamers (aka not-so-random hexamers). Preferably, the template particles are linked to the capture oligos that include one or more primer binding sequences. However, in other aspects, the capture oligos may be released from the template particles prior to attachment with the target fragment.

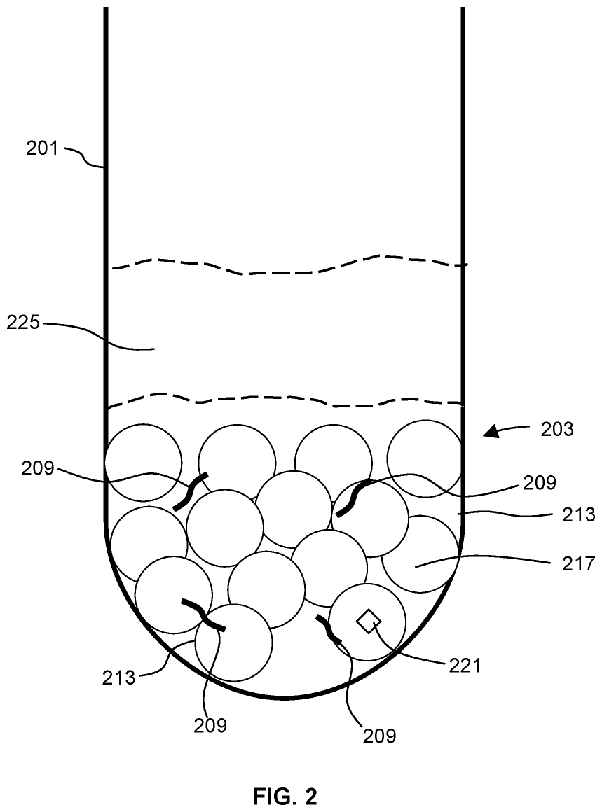

FIG. 2 shows a vessel containing nucleic acids and template particles before vortexing. The vessel 201 includes a mixture of cfDNA 209 and template particles 217 inside an aqueous fluid 213 with an oil overlay. Shown, is an illustration of the vessel 201 after the combining step 109 of method 101. The aqueous fluid 213 may include certain reagents, such as, reagents for preserving samples of nucleic acids, e.g., EDTA, or for nucleic acid synthesis, such as, reagents for PCR. In some embodiments, the reagents may be provided by template particles 217. Accordingly, template particles 217 may include one or more compartments 221 containing the reagents, which are releasable from the compartments 221 in response to an external stimulus, such as, for example, heat, osmotic pressure, or an enzyme. Reagents may include nucleic acid synthesis reagents, such as, for example, a polymerase, primers, dNTPs, or buffers. In addition, the vessel 201 further includes a second fluid 225 that is immiscible with the first fluid, e.g., an oil.

In some aspects, generating the template particles-based monodisperse droplets involves shearing two liquid phases. The liquid phase comprising template particles and nucleic acids is the aqueous phase and, in some embodiments, the aqueous phase may further include reagents selected from, for example, buffers, salts, lytic enzymes (e.g. proteinase k) and/or other lytic reagents (e. g. Triton X-100, Tween-20, IGEPAL, bm 135, or combinations thereof), nucleic acid synthesis reagents e.g. nucleic acid amplification reagents. The second phase is a continuous phase and may be an immiscible oil such as fluorocarbon oil, a silicone oil, or a hydrocarbon oil, or a combination thereof. In some embodiments, the fluid may comprise reagents such as surfactants (e.g. octylphenol ethoxylate and/or octylphenoxypolyethoxyethanol), reducing agents (e.g. DTT, beta mercaptoethanol, or combinations thereof). For example, see Hatori et. al., Anal. Chem., 2018 (90):9813-9820, which is incorporated by reference.

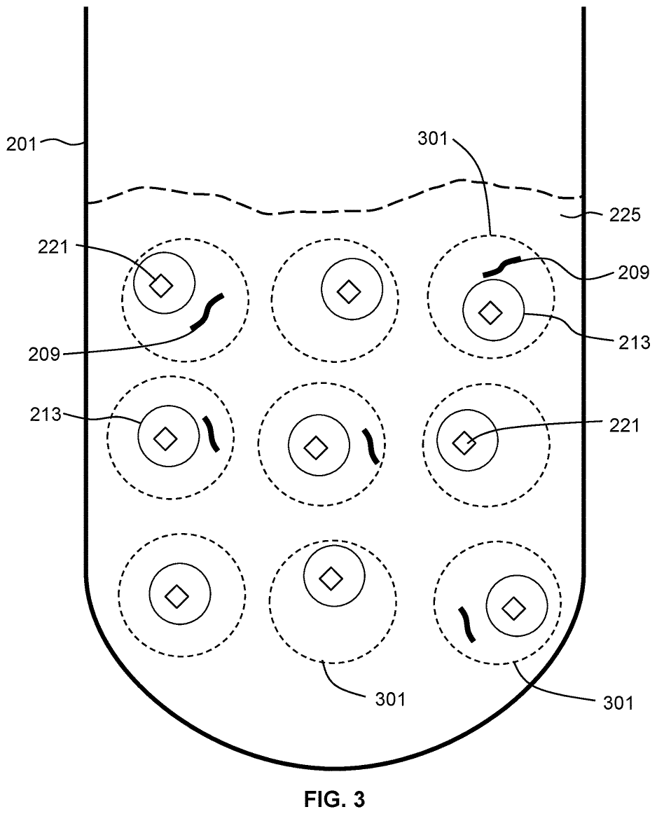

FIG. 3 shows a vessel 229 containing nucleic acids 209 and template particles 217 inside droplets. The vessel 229 includes a plurality of monodisperse droplets 301, some of which contain a single fragment of nucleic acid, i.e., cfDNA 209, and a temple particle 213. A person of skill in the art will recognize that not all of the droplets 301 generated according to aspects of the invention will necessarily include a single one of the fragments 209 and a single one of the template particles 217. In some instances, a droplet 301 may include more than one, or none, the fragments 209 or template particles 217. Droplets that do not contain one of each a fragment 209 and a template particle 217 may be removed from the vessel 201, destroyed, or otherwise ignored. In some instances, template particles 217 may be formulated so as to have a positive surface charge, or an increased positive surface charge. Such materials may be without limitation poly-lysine or polyethyleneimine, or combinations thereof. This increases the probability of an association between the template particle 217 and the cfDNA 209, which is negatively charged.

Other strategies aimed to increase the chances of an association with a template particle 217 include creating specific template particle 217 geometries. For example, in some embodiments, the template particles may have a general spherical shape but the shape may contain features such as flat surfaces, craters, grooves, protrusions, and other irregularities in the spherical shape that enhance the associate between the template particle 217 and the fragment of cfDNA 209 thereby improving the probability that each monodisperse droplet will contain one fragment of cfDNA 209.

Template particles include compartments, such as, micro-compartments, or internal compartments, which may contain additional components and/or reagents, e.g., additional components and/or reagents that may be releasable into monodisperse droplets 301. Reagents may include, for example, a DNA polymerase.

Template particles of the present disclosure may include a plurality of capture probes. Generally, the capture probe is an oligonucleotide. The capture probes may be attached to the template particle's material, e.g. hydrogel material, via covalent acrylic linkages. In some embodiments, the capture probes are acrydite-modified on their 5′ end (linker region). Generally, acrydite-modified oligonucleotides can be incorporated, stoichiometrically, into hydrogels such as polyacrylamide, using standard free radical polymerization chemistry, where the double bond in the acrydite group reacts with other activated double bond containing compounds such as acrylamide. Specifically, copolymerization of the acrydite-modified capture probes with acrylamide including a crosslinker, e.g. N,N′-methylenebis, will result in a crosslinked gel material comprising covalently attached capture probes. In some other embodiments, the capture probes comprise acrylate terminated hydrocarbon linker and combining the said capture probes with a template particle will cause their attachment to the template particle.

The capture probe may comprise one or more of a primer sequence, a barcode unique to each droplet, a unique molecule identifier (UMI), and a capture sequence.

Primer sequences may comprise a binding site, for example a primer sequence that would be expected to hybridize to a complementary sequence, if present, on any target nucleic acid molecule and provide an initiation site for a reaction, for example an elongation or polymerization reaction. The primer sequence may also be a “universal” primer sequence, i.e. a sequence that is complimentary to nucleotide sequences that are very common for a particular set of nucleic acid fragments. The primer sequences used may be P5 and P7 primers as provided by Illumin, Inc., San Diego, Calif. The primer sequence may also allow the capture probe to bind to a solid support, such as a template particle.

By providing capture probes comprising the barcode unique to each droplet, the capture probes may be use to tag the nucleic molecules inside droplets with the barcode.

Unique molecule identifiers (UMIs) are a type of barcode that may be provided to nucleic acid molecules in a sample to make each nucleic acid molecule, together with its barcode, unique, or nearly unique. This is accomplished by adding, e.g. by ligation, one or more UMIs to the end or ends of each nucleic acid molecule such that it is unlikely that any two previously identical nucleic acid molecules, together with their UMIs, have the same sequence. By selecting an appropriate number of UMIs, every nucleic acid molecule in the sample, together with its UMI, will be unique or nearly unique. One strategy for doing so is to provide to a sample of nucleic acid molecules a number of UMIs in excess of the number of starting nucleic acid molecules in the sample. By doing so, each starting nucleic molecule will be provided with different UMIs, therefore making each molecule together with its UMIs unique. However, the number of UMIs provided may be as few as the number of identical nucleic acid molecules in the original sample. For example, where no more than six nucleic acid molecules in a sample are likely to be identical, as few as six different UMIs may be provided, regardless of the number of starting nucleic acid molecules.

UMIs are advantageous in that they can be used to correct for errors created during amplification, such as amplification bias or incorrect base pairing during amplification. For example, when using UMIs, because every nucleic acid molecule in a sample together with its UMI or UMIs is unique or nearly unique, after amplification and sequencing, molecules with identical sequences may be considered to refer to the same starting nucleic acid molecule, thereby reducing amplification bias. Methods for error correction using UMIs are described in Karlsson et al., 2016, Counting Molecules in cell-free DNA and single cells RNA″, Karolinska Institutet, Stockholm Sweden, incorporated herein by reference. Capture sequences used in capture probes are advantageous for targeting gene-specific nucleotide sequences, for example nucleotide sequences known to be associated with a particular cancer genotype or phenotype. In such methods, the target nucleic acid sequence, if present, attaches to the template particle by hybridizing to the capture sequence.

In some embodiments, amplified target nucleic acids may be analyzed by sequencing, which may be performed by methods known in the art. For example, see, generally, Quail, et al., 2012, A tale of three next generation sequencing platforms: comparison of Ion Torrent, Pacific Biosciences and Illumina MiSeq sequencers, BMC Genomics 13:341. Nucleic acid sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

The conventional pipeline for processing sequencing data includes generating FASTQ-format files that contain reads sequenced from a next generation sequencing platform, and aligning these reads to an annotated reference genome. These steps are routinely performed using known computer algorithms, which a person skilled in the art will recognize can be used for executing steps of the present invention. For example, see Kukurba, Cold Spring Harb Protoc, 2015 (11):951-969, incorporated by reference.

The sequence reads may be analyzed to identify mutations. For example, sequence reads derived from a fragment of amplified ctDNA may be analyzed to identify small mutations such as polymorphisms or small indels. To identify small mutations, reads may be mapped to a reference using assembly and alignment techniques known in the art or developed for use in the workflow. Various strategies for the alignment and assembly of sequence reads, including the assembly of sequence reads into contigs, are described in detail in U.S. Pat. No. 8,209,130, incorporated herein by reference. Strategies may include (i) assembling reads into contigs and aligning the contigs to a reference; (ii) aligning individual reads to the reference; or (iv) other strategies known to be developed or known in the art. Sequence assembly can be done by methods known in the art including reference-based assemblies, de novo assemblies, assembly by alignment, or combination methods. Sequence assembly is described in U.S. Pat. Nos. 8,165,821; 7,809,509; 6,223,128; U.S. Pub. 2011/0257889; and U.S. Pub. 2009/0318310, the contents of each of which are hereby incorporated by reference in their entirety. Sequence assembly or mapping may employ assembly steps, alignment steps, or both. Assembly can be implemented, for example, by the program ‘The Short Sequence Assembly by k-mer search and 3'read Extension’ (SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501, incorporated by reference). SSAKE cycles through a table of reads and searches a prefix tree for the longest possible overlap between any two sequences. SSAKE clusters reads into contigs.

What is claimed is:

1. A method for amplifying cfDNA, the method comprising:
obtaining a sample comprising a cfDNA fragment in blood or plasma;
combining template particles and the sample in an aqueous fluid within a vessel;
adding a second fluid to the vessel;
vortexing the vessel to form a plurality of monodisperse droplets simultaneously, wherein the cfDNA fragment is isolated within one of the droplets; and
amplifying the cfDNA fragment inside the droplet.

2. The method of claim 1, wherein the vessel is a blood collection tube.

3. The method of claim 1, wherein the cfDNA fragment is ctDNA.

4. The method of claim 3, wherein amplifying includes generating a plurality of amplicons that can be analyzed to provide genetic information from the subject.

5. The method of claim 4, wherein the genetic information describes one or more mutations in the subject.

6. The method of claim 5, wherein the ctDNA includes a mutation specific to a tumor.

7. The method of claim 6, wherein the amplicons are analyzed by sequencing.

8. The method of claim 1, wherein amplifying the target cfDNA fragment includes barcoding the cfDNA fragment.

9. The method of claim 1, wherein vortexing the vessel comprises placing the vessel onto a vortexer.

10. The method of claim 1, wherein the template particles comprise one or more compartments.

11. The method of claim 10, wherein the one or more compartments contain a reagent releasable from the one or more compartments into the monodisperse droplet.

12. The method of claim 11, wherein the reagent is a DNA polymerase.

13. The method of claim 12, wherein the reagent is released from the one or more compartments in response to an external stimulus.

14. The method of claim 13, wherein the template particles comprise hydrogel selected from agarose, alginate, a polyethylene glycol (PEG), a polyacrylamide (PAA), acrylate, acrylamide/bisacrylamide copolymer matrix, azide-modified PEG, poly-lysine, polyethyleneimine, or any combination thereof.

15. The method of claim 1, wherein amplifying includes multiple displacement amplification.

16. A method for amplifying nucleic acid molecules present at a low concentration, the method comprising:
combining template particles and nucleic acid molecules in an aqueous fluid within a vessel, wherein the concentration of the nucleic acid molecules inside the vessel is below 1 ng per μl;
adding a second fluid to the vessel;
vortexing the vessel to form a plurality of monodisperse droplets simultaneously, wherein each of the nucleic acid molecules are contained within a separate one of the droplets; and
amplifying the nucleic acid molecules contained inside the droplets.

17. The method of claim 16, wherein the second fluid is immiscible with the first fluid.

18. The method of claim 17, wherein after combining the nucleic acid molecules with the template particles the concentration of nucleic acid molecules is below 1 pg per μl.
